(19) United States
(12) Patent Application Publication
(10) Pub. No.: US 2017/0055960 A1
(43) Pub. Date: Mar. 2, 2017

(54) Blade Holder

(71) Applicants: Albert A. Kattine, Nashville, TN (US); David H. Horowitz, Nashville, TN (US)

(72) Inventors: Albert A. Kattine, Nashville, TN (US); David H. Horowitz, Nashville, TN (US)

(21) Appl. No.: 14/840,836

(22) Filed: Aug. 31, 2015

(51) Int. Cl. A61B 10/02

(52) U.S. Cl. CPC A61B 10/0233

(57) ABSTRACT

A biopsy blade holder is provided that includes a substantially U-shaped body with a base, first and second opposing elongated members, first and second attachment structures and a restrictor. The elongated members extend from the base and diverge away from each other to first and second distal ends. A blade is inserted in the structures or fixedly attached near the distal ends. The restrictor is removably mounted to the exterior surfaces of the elongated members and adjusts and affixes the distance between the elongated members. Moving the restrictor towards the distal ends causes the blade to flex outwardly and adjusts the length of the blade edge that contacts the surface of a patient's skin.

### FIELD OF THE INVENTION

The present invention is a device for holding a blade used for controlled cutting or slicing. In particular, the present invention relates to a device that adjustably holds a blade used to perform a biopsy or to remove lesions so that the shape and the length of the blade that contacts a patient's skin can be easily changed.

### BACKGROUND OF INVENTION

A biopsy is the medical removal of tissue from a living subject to determine the presence or extent of a disease. Skin biopsies are useful for patients with dermatologic disorders that cannot be diagnosed on the basis of a clinical examination alone. The removed tissue is generally examined under a microscope by a pathologist and/or analyzed chemically at an offsite location. When an entire lump or suspicious area is removed, the procedure is called an excisional biopsy. When only a sample of tissue is removed with preservation of the histological architecture of the tissue's cells, the procedure is called an incisional biopsy or core biopsy. Biopsies are most commonly performed for insight into possible cancerous and inflammatory conditions. At times a skin growth is removed because it is irritated, a nuisance or unsightly. Redundant areas of skin and excess tissue may also be removed for functional or cosmetic purposes.

Typically, a biopsy or lesion removal is performed by a shave technique that can use a variety of different devices, including a sharp dermal curette, a flexible metal razor blade, a scissors, an electrosurgical wire loop and a No. 15 scalpel blade. Standard double edge razor blades have been used to perform the shave technique or “razor blade surgery” by manually breaking a double edge razor blade in half along its length. The modified “single edge blade” was then held between the fingers to perform the biopsy or lesion removal. A shave biopsy is simple and can be performed by a physician or medical technician in an office setting. Shave biopsies and removals are recommended for raised or pedunculated lesions and enable easy removal of growths on protruding or convex surfaces (e.g., the pinna and the nose), as well as numerous superficial lesions (e.g., seborrheic keratoses, verrucae, nevi, actinic keratoses, and angiomas). Shave biopsies and removals also yield good cosmetic results, since the wound edges can be contoured to minimize scarring. The wounds heal by secondary intention, and sutures are not necessary.

Most shave biopsies and removals are performed at the level of the dermis. Superficial skin lesions can be excised completely by cutting sufficiently deep, but the deeper the incision, the greater the likelihood of scarring. Proper shave technique often balances the need to remove all cells of a lesion with the desire not to cut too deeply. The cosmetic results can often be predicted by the skin surface surrounding the lesion. When this procedure is performed on concave surfaces of the nose, eye, ear, and temple, the wounds often heal with nearly imperceptible scars. Shave biopsies and removals performed on convex surfaces may result in round, hypo-pigmented scars.

The devices that are currently being used to perform shave biopsies and removals do not provide the dermatologist with sufficient control of the cutting blade to remove a precise surface area of the targeted lesion at a precise depth. For example, scalpels and scalpel blades have been used, but there is insufficient flexibility due to the rigidity of the blade and the handle. Therefore, there is a need for a biopsy blade holder that provides the dermatologist with a more flexible cutting blade with increased control and an easy means to adjust the shape and the length of the cutting blade that contacts the patient's skin.

### SUMMARY OF THE INVENTION

In accordance with the present invention, a biopsy blade holder is provided. The biopsy blade holder comprises, consists of or consists essentially of a substantially U-shaped body and a restrictor. The U-shaped body has a base, first and second opposing elongated members, and first and second attachment structures. Each of the first and second elongated members (also referred to herein as first and second legs) has interior and exterior surfaces and each divergently extends from the base to first and second distal ends. The first and second opposing elongated members are separated by a distance and the distance increases as the members extend from the base to the distal ends. The first and second attachment structures are located near the first and second distal ends, respectively, of the elongated members and are adapted to secure a blade in the blade holder.

The restrictor is removably mounted on the exterior surfaces of the first and second elongated members and adjusts and affixes the distance between the elongated members. The restrictor includes first and second end walls having first and second interior surfaces and at least one side wall (the restrictor can also have first and second side walls) that connects the first and second end walls and defines an opening. The at least one side wall has a top edge and a bottom edge and the end walls preferably extend outwardly between the top edge and the bottom edge, which results in the side wall or walls having a trapezoidal shape. The first and second interior surfaces of the end walls contact the first and second exterior surfaces of the elongated members, respectively. The first and second end walls can have at least one ridge that extends from the interior surface and is perpendicular to the side wall. The base is inserted through the opening in the restrictor and moved towards the distal ends of the elongated members. The exterior surface of each of the elongated members has two or more transverse ridges that are engaged by the at least one ridge on the interior surface of each of the end walls to secure the restrictor to the elongated members and prevent the elongated members from moving apart (i.e., prevent the distance between the elongated members from increasing).

The blade holder is designed to hold a blade that is removably or fixedly (i.e., permanently) attached to the first and second attachment structures. When a blade is removably attached, it can be removed and replaced with a new blade. When a blade is fixedly attached, the blade holder is designed to be disposable and the blade is not intended to be removed. The blade can be a rigid or a flexible blade; however, a flexible blade is preferred because it allows the user to adjust the cutting surface of the blade by pressing on the exterior surfaces of the elongated members and/or adjusting the position of the restrictor to move the elongated members together. The distal ends of each of the opposing elongated members can extend outwardly from the elongated members and form first and second heels on the interior surfaces of the elongated members opposite the distal ends. The attachment structures are formed in the first and second heels and are opposite to each other. Preferably, the attachment structures are formed by first and second slots that are adapted to receive the first and second ends of a blade. However, other means can be used for retaining the blade, such as pins, rivets, clips or screws.

In another embodiment, the blade holder comprises, consists of or consists essentially of a body having first and second opposing elongated members, first and second attachment structures and a restrictor. The first and second elongated members have first and second proximal ends, first and second exterior surfaces and first and second opposing interior surfaces. The proximal ends are connected at a base and the elongated members divergently extend from the proximal ends to first and second distal ends. The first and second elongated members are separated by a distance and the distance increases as the members extend from the proximal ends to the distal ends. The first and second attachment structures are located at the first and second distal ends, respectively, and are adapted to secure a blade to the blade holder.

The restrictor is removably mounted on the first and second elongated members and adjusts and affixes the distance between the elongated members. The restrictor includes first and second end walls having first and second interior surfaces and at least one side wall (preferably first and second side walls) that connects the first and second end walls and defines an opening. The first and second interior surfaces of the end walls contact the first and second exterior surfaces of the elongated members, respectively. The at least one side wall has a top edge and a bottom edge and the end walls extend outwardly between the top edge and the bottom edge, which results in the side wall or walls having a trapezoidal shape. The base and proximal ends of the elongated members are inserted through the opening in the restrictor.

The interior surfaces of the first and second end walls can have at least one ridge extending between and perpendicular to the side walls. The exterior surfaces of the elongated members can have a plurality of transverse ridges that are engaged by the at least one ridge on the interior surfaces of the end walls to secure the restrictor to the elongated members and prevent the elongated members from moving apart.

The blade can be substantially rigid or flexible and is removably or permanently attached to the first and second attachment structures. The distal ends of each of the opposing elongated members extend inwardly so that the attachment structures are directly opposite to each other. In another embodiment, the distal ends extend outwardly to form first and second heels in the opposing elongated members. The attachment structures are formed in the distal ends or in the heels and can be first and second slots that are adapted to receive the opposing ends of the blade or other means can be used for retaining the blade, such as pins, rivets, clips or screws.

### BRIEF DESCRIPTION OF THE FIGURES

The preferred embodiments of the biopsy blade holder of the present invention, as well as other objects, features and advantages of this invention, will be apparent from the accompanying drawings wherein:

FIG. 1 is a perspective view of a first embodiment of the biopsy blade holder of the present invention.

FIG. 2 is a detailed view of the distal end of the biopsy blade holder shown in FIG. 1.

FIG. 3 is a side view of the biopsy blade holder shown in FIG. 1 with the restrictor removed from the biopsy blade holder.

FIG. 4 is a perspective view of a second embodiment of the biopsy blade holder of the present invention with a blade attached to the distal ends of the legs.

FIG. 5 is a side view of the biopsy blade holder shown in FIG. 1 being held between the thumb and forefinger of a user.

FIGS. 6A-E show a razor blade cut in half and secured in the blade holder.

### DETAILED DESCRIPTION OF THE INVENTION

The present invention is a biopsy blade holder that is used to remove moles, warts, calluses, skin tags, keratoses, or other lesions from a patient either by means of biopsy, shave removal, saucerization or excision. The removed tissue can then be tested to determine whether it is benign or a malignant lesion (e.g., cancer) that would require additional treatment. The biopsy blade holder can also be used for cosmetic purposes, such as reshaping noses that are deformed by rhinophyma. The biopsy blade holder includes a U-shaped body with first and second elongated members that are joined at the proximal ends and extend therefrom to first and second distal ends; preferably, the elongated members are joined in a base. A restrictor is removably attached to the two elongated members and moving the restrictor between the proximal ends and distal ends of the blade holder adjusts the distance between the elongated members. When a flexible blade is inserted into the slots located near the distal ends, the position of the restrictor determines the configuration of the blade. As the restrictor is moved towards the distal ends, the elongated members move closer together and the shape of the blade changes from straight to arcuate with the blade bowing away from the blade holder in a convex configuration which adjusts the length of the blade edge that contacts the patient's skin.

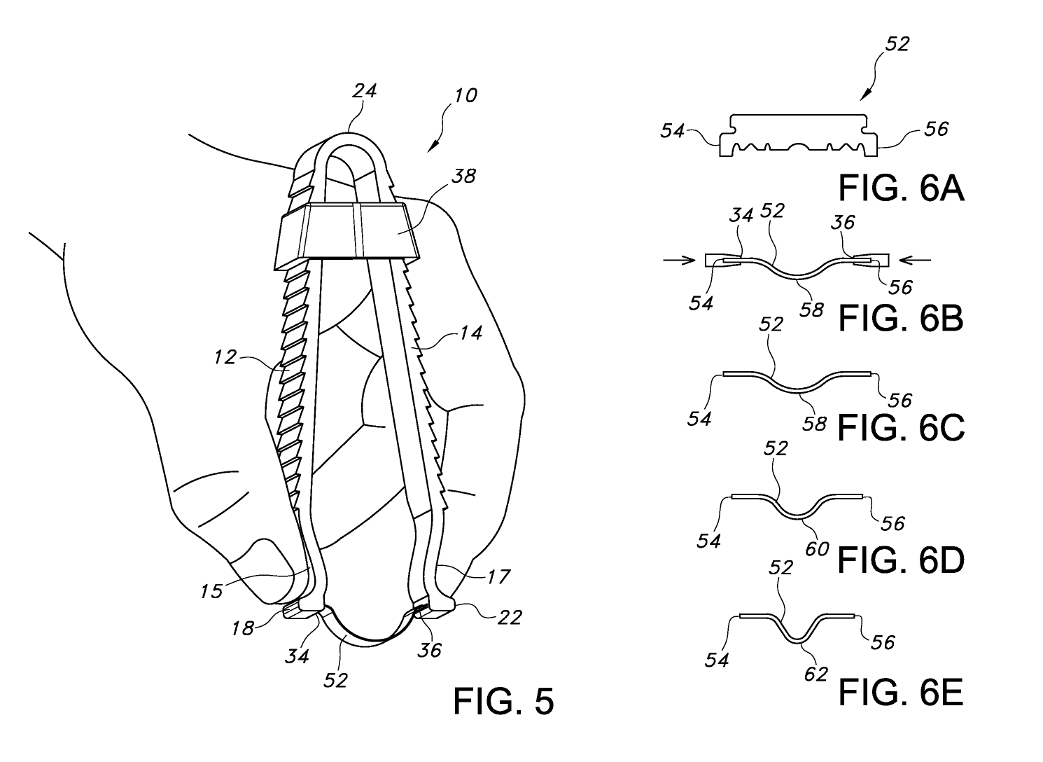

The two elongated members (also referred to herein as “legs”) of the biopsy blade holder can be pushed together by the user to flex the blade (e.g., a razor blade) so that smaller growths can be cut off. Once the blade is in the desired configuration, the restrictor can be used to maintain the position of the elongated members and the shape of the blade. Typically, the exterior surfaces of the distal ends of the elongated members are gripped by the thumb and the forefinger or middle finger or both of the user's hand (see FIG. 5). After a blade is fitted into the lower end of the device, a restrictor or sleeve around the two legs is pushed down along the exterior surfaces of the legs to move them closer together and hold the blade in place. In one embodiment, the blade is permanently attached to the distal ends of the legs using pins, rivets, clips or screws or by forming the blade in the legs when the blade holder is made of plastic (e.g., in a molding or extrusion process) and is designed to be disposable.

In one embodiment, the blade holder is designed so that a conventional double edge razor blade can be used. The double edge razor blade is cut in half along its longitudinal axis and only one of the two edges is used in the blade holder. To install the halved blade in the blade holder, the restrictor is moved towards the proximal end of the blade holder to allow the legs to be spread apart. The opposing ends of the blade are then inserted into slots in the distal ends of the legs. After the blade is installed, the restrictor is moved down the legs towards the distal ends to secure the blade in the blade holder. Pressing the legs together causes the blade to bend in a convex configuration that reduces the length of the blade edge that contacts the surface of the patient's skin.

The ability to flex the blade provides the advantage of being able to achieve different depths of cutting with a consistent and even cut. Also, the flexed blade can more easily conform to body contours (convex and concave surfaces) and provides access to areas of various size limitations and complexity due to the ability to bend the blade. The blade is secured in place by inserting the ends of the blade (e.g., a halved double edge razor blade—see FIG. 6A) into the slots at the ends of the elongated members/legs of the holder. The depth of the slots is selected to ensure that the blade remains in the holder and does not spring out under high tension when the elongated members are pressed together. Although the blade holder is described for use in dermatological procedures, it is not limited to such usage and may be used for any task that requires controlled cutting or slicing of an object or organism with a sharp blade.

The preferred material for the blade holder is a disposable plastic which would be sterilized and packaged for the initial use. Preferably, the blade holder is a single use all-in-one disposable device that includes the blade holder as well as the blade. In this embodiment, the blade is preferably permanently affixed to the distal ends of the plastic holder to eliminate the possibility of the blade “springing out” of the holder. This technology is well known and used extensively to manufacture single use razors. In another embodiment, the blade holder can be reused by practitioners after being cleaned; preferably, using cold sterilization procedures. For embodiments of the blade holder intended to be a “permanent” or reusable device, which could be continuously reused and autoclaved, the preferred material is surgical steel or similar materials used for medical instruments. However, the blade holder and blade are not required to be sterile; the blades can merely be “clean” since the skin surface is full of bacteria.

As used herein, the term “U-shaped body” refers to the embodiment of the device wherein a curved base section joins the proximal ends of the two elongated members. The base and the elongated members are preferably formed as a unitary structure and the elongated members divergently extend to distal ends. The distance between the members increases as the elongated members extend towards the distal ends.

As used herein, the term “attachment structure” refers to the structures near the distal end of each elongated member that is used to affix the end of the blade in the blade holder. A variety of different attachment structures can be used. The attachment structures and the ends of the blade can have “keyed” configurations. Also, when the distal ends extend away from the elongated members to form a heel on each of the interior surfaces, the section between the heel and the distal end can be hinged or slide open and closed to install the blade. Preferably, the attachment structure is a slot. The elongated members are spread apart and the ends of a blade are inserted into the slots in each of the elongated members. The restrictor is then moved towards the distal ends to secure the blade in the blade holder.

As used herein, the term “blade” refers to a substantially flat, thin device with at least one sharp edge that is used for cutting. For example, a single edge razor blade or a double edge razor blade that is cut in half along its longitudinal axis, i.e. between its two ends. The term “flexible blade” refers to a blade that can easily be bent by applying pressure to its opposing ends and the term “rigid blade” refers to a blade that cannot be easily bent by applying pressure to its opposing ends.

As used herein, the term “restrictor” refers to a device having first and second end walls connected by at least one side wall that is configured to move up and down around the exterior surfaces of the elongated members to control the distance between the elongated members. The exterior surfaces of the elongated members and the interior surfaces of the end walls of the restrictor can have transverse ridges that engage to lock the restrictor into position. The side wall of the restrictor can have a trapezoidal shape with the distance between the end walls increasing between the top edge and bottom edge of the side wall so that the slope of the end walls is similar to the slope of the elongated members.

As used herein with respect to the exterior surfaces of the elongated members and the interior surfaces of the end walls of the restrictor, the term “ridge” refers to one or more raised portions extending from the surface in a direction transverse to the longitudinal axis of the elongated members and perpendicular to side walls of the restrictor. When the restrictor is positioned over the elongated members, the ridges on the interior surfaces of the end walls of the restrictor and the ridges on the exterior surfaces of the elongated members engage to lock the restrictor into position with the distance between the distal ends of the elongated members fixed. The restrictor is repositioned by pressing the elongate members together to disengage the ridges of the respective surfaces. After the position of the restrictor is reset, the elongated members are released and the restrictor is once more affixed, i.e., locked in place. The ridges can have a variety of different configurations; for example, the apex of the ridges can be rounded, triangular or flat. The shape of the ridges is not intended to limit the invention and the only requirement for the ridges is that they securely engage the restrictor on the elongated members and they can be easily disengaged to reposition the restrictor.

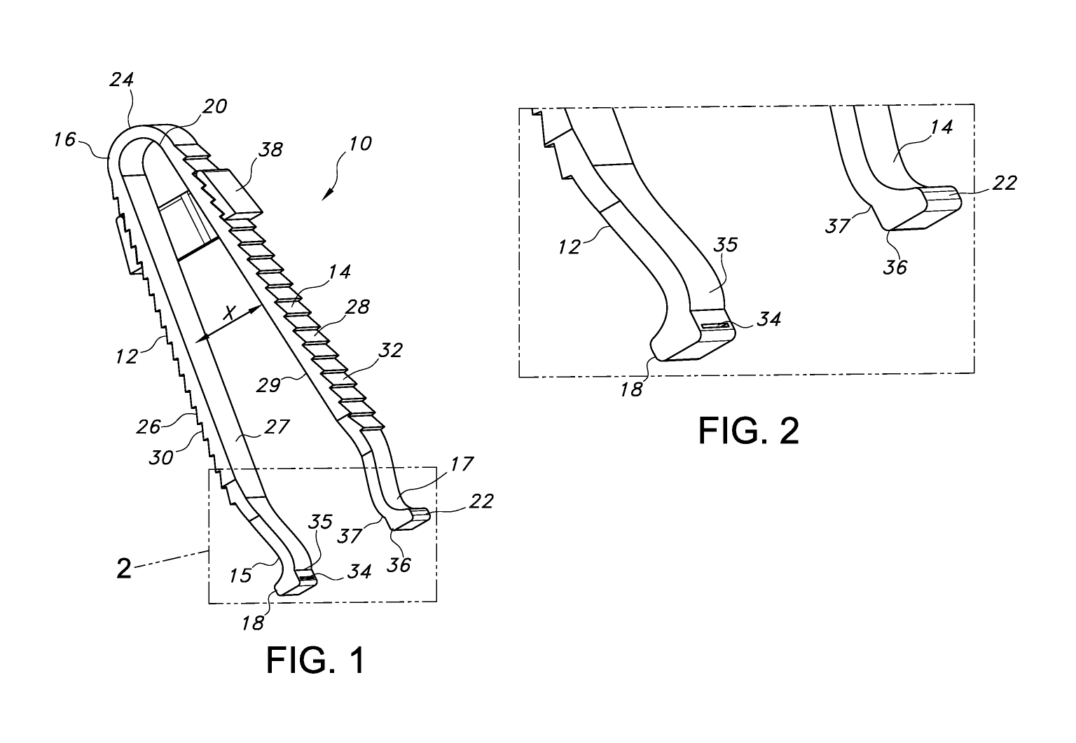

Referring now to the drawings, FIG. 1 shows an embodiment of the biopsy blade holder 10 having first and second opposing elongated members 12, 14 (also referred to as legs) that extend between first and second proximal ends 16, 20 and first and second distal ends 18, 22. The proximal ends 16, 20 are joined at a base 24 and the distance (X) between the elongated members 12, 14 increases as they extend from the base 24. Before reaching the distal ends 18, 22, the elongated members 12, 14 curve inwardly to form recesses 15, 17 that are used by the practitioner to grasp the blade holder 10. The elongated members 12, 14 have exterior surfaces 26, 28 with a plurality of ridges 30, 32 extending therefrom and interior surfaces 27, 29. The distal ends 18, 22 of the elongated members 12, 14 extend outwardly and form first and second heels 35, 37 on the opposing interior surfaces 27, 29 of the elongated members 12, 14. As shown in FIG. 2, the attachment structures are formed by slots 34, 36 in the heels 35, 37.

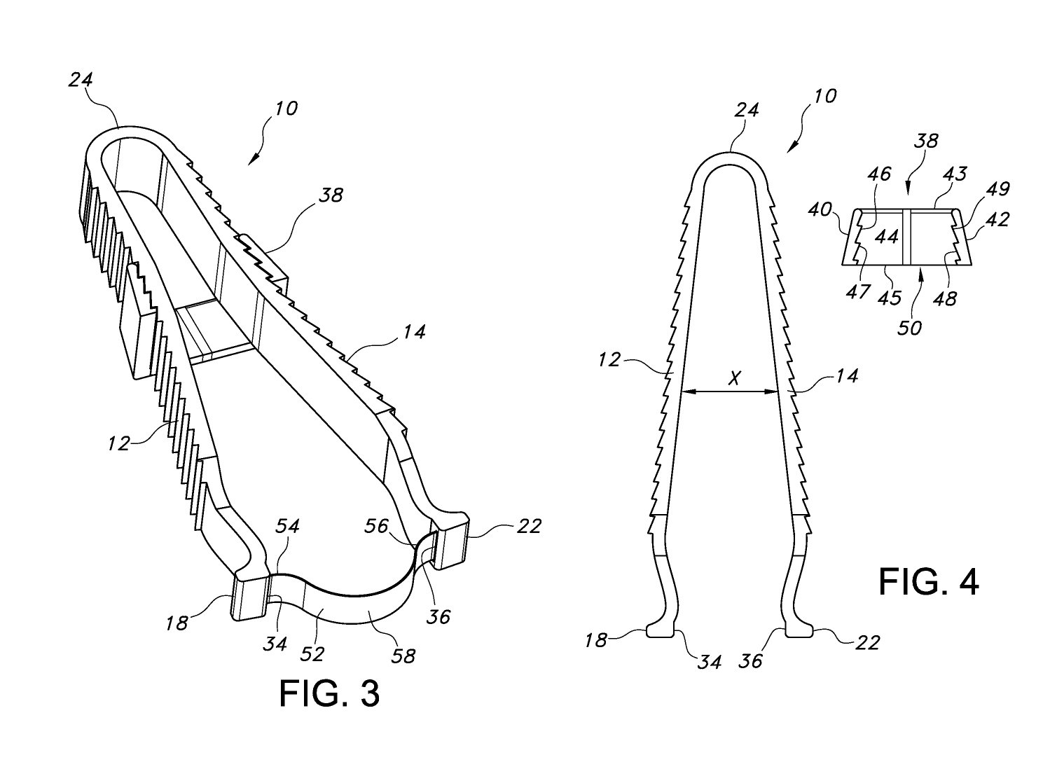

A restrictor 38 fits over the base 24 of the blade holder 10 and regulates the distance (X) between the elongated members 12, 14. As shown in FIG. 3, the restrictor 38 has first and second end walls 40, 42 connected by a side wall 44 having a top edge 43 and a bottom edge 45. The first and second end walls 40, 42 slope outwardly between the top edge 43 and the bottom edge 45 and have first and second interior surfaces 46, 48 with one or more ridges 47, 49, respectively, extending from the interior surfaces 46, 48. The first and second end walls 40, 42 and the side wall 44 define an opening 50 that receives the base 24. The one or more ridges 47, 49 on the interior surfaces 46, 48 of the end walls 40, 42 engage the plurality of ridges 30, 32 extending from the exterior surfaces 26, 28 of the first and second elongated members 12, 14 to lock the restrictor 38 onto the blade holder 10. This prevents the restrictor 38 from moving and maintains a fixed distance (X) between the elongated members 12, 14. To reposition the restrictor 38, the user pushes the elongated members 12, 14 together to disengage the ridges 47, 49 on the interior surfaces 46, 48 of the end walls 40, 42 from the ridges 30, 32 on the exterior surfaces 26, 28 of the elongated members 12, 14. Once the ridges 30, 32 on the restrictor and the ridges 47, 49 on the elongated members 12,14 are disengaged, the restrictor 38 can be freely moved up and down the elongated members 12, 14 to a new location to adjust the distance (X) between the members 12, 14.

FIG. 4 shows a blade 52 installed in the blade holder 10 with the opposing ends 54, 56 positioned in slots 34, 36 of the opposing elongated members 12, 14. The elongated members 12, 14 are close together so that the blade 52 is in a flexed position and the intermediate portion of the blade 58 extends convexly, i.e., outwardly and away from the blade holder 10. The configuration of the blade 52 is determined by the distance (X) between the elongated members 12, 14 and, after the user adjusts the blade 52 to the desired configuration by pressing the elongated members 12, 14 together, the restrictor 38 can be moved into a position and affixed to the elongated members 12, 14 in order to maintain the desired configuration.

FIG. 5 shows a user holding the biopsy blade holder 10 with a thumb against the first recess 15 in the first elongated member 12 and a forefinger against the second recess 17 in the second elongated member 14. The restrictor 38 is positioned so that the blade 52 is bowed outwardly. In this configuration, the user can use the blade 52 to cut a growth from difficult to access locations on a patient's skin.

FIGS. 6A-E show the blade in different configurations. FIG. 6A shows a double edge razor blade 52 that is cut in half between ends 54, 56. In FIG. 6B, a blade 52 is inserted in the slots 34, 36 that form the attachment structure. The arrows on either side indicate the direction of the pressure that is applied when the elongated members 12, 14 (see FIG. 5) are pressed together with the intermediate section 58 of the blade 52 flexing outwardly. This same configuration of the intermediate section 58 of the blade 52 is shown in FIG. 6C. FIG. 6D shows the configuration of the intermediate section 60 of the blade 52 as the elongated members 12, 14 are pressed closer together and FIG. 6E shows the configuration of the intermediate section 62 of the blade 52 as additional pressure is applied to the elongated members 12, 14. The user adjusts the blade 52 to a desired configuration and then locks the elongated members 12, 14 in position by adjusting the restrictor 38 (see FIG. 5) to maintain the configuration.

Thus, while there have been described the preferred embodiments of the present invention, those skilled in the art will realize that other embodiments can be made without departing from the spirit of the invention, and it is intended to include all such further modifications and changes as come within the true scope of the claims set forth herein.

We claim:

1. A blade holder comprising:
a substantially U-shaped body comprising:
a base;
first and second opposing elongated members extending divergently from the base and each having interior and exterior surfaces and first and second distal ends, wherein the first and second opposing elongated members are separated by a distance and the distance increases as the members extend from the base to the distal ends; and
first and second attachment structures located near the first and second distal ends, respectively, wherein the attachment structures are adapted to secure a blade in the blade holder; and
a restrictor removably mounted on the exterior surfaces of the first and second opposing elongated members, wherein the restrictor adjusts and affixes the distance between the first and second opposing elongated members.

2. The blade holder according to claim 1, wherein the restrictor comprises first and second end walls having first and second interior surfaces and at least one side wall connecting the first and second end walls and defining an opening, and wherein the first and second interior surfaces of the end walls contact the first and second exterior surfaces of the elongated members, respectively.

3. The blade holder according to claim 2, wherein the restrictor has a first side wall and a second side wall, and wherein the base is inserted through the opening in the restrictor.

4. The blade holder according to claim 1, wherein each of the first and second end walls has at least one ridge extending from the interior surface, and wherein the at least one ridge is perpendicular to the at least one side wall.

5. The blade holder according to claim 4, wherein the exterior surface of each of the elongated members has two or more transverse ridges, and wherein the ridges are engaged by the at least one ridge on the interior surface of each of the end walls to secure the restrictor to the elongated members and prevent the elongated members from moving apart.

6. The blade holder according to claim 1, wherein a blade is removably attached to the first and second attachment structures.

7. The blade holder according to claim 1, wherein a flexible blade is removably attached to the first and second attachment structures.

8. The blade holder according to claim 1, wherein a blade is fixedly attached to the first and second attachment structures.

9. The blade holder according to claim 1, wherein a flexible blade is fixedly attached to the first and second attachment structures.

10. The blade holder according to claim 1, wherein the distal ends extend outwardly from the elongated members and form first and second heels on the interior surfaces of the elongated members opposite the distal ends, and wherein the attachment structures are formed in the first and second heels and are opposite to each other.

11. The blade holder according to claim 1, wherein the attachment structures are formed by first and second slots that are adapted to receive a blade having first and second ends.

12. A blade holder comprising:
first and second elongated members having first and second proximal ends, first and second exterior surfaces and first and second opposing interior surfaces, wherein the proximal ends are connected and the elongated members divergently extend from the proximal ends to first and second distal ends, wherein the first and second elongated members are separated by a distance and the distance increases as the members extend from the proximal ends to the distal ends;
first and second attachment structures located near the first and second distal ends, respectively, wherein the attachment structures are adapted to secure a blade to the blade holder; and
a restrictor removably mounted on the first and second elongated members, wherein the restrictor adjusts and affixes the distance between the first and second elongated members.

13. The blade holder according to claim 12, wherein the restrictor comprises first and second end walls having first and second interior surfaces and at least one side wall connecting the first and second end walls and defining an opening, and wherein the first and second interior surfaces of the end walls contact the first and second exterior surfaces of the elongated members, respectively.

14. The blade holder according to claim 13, wherein the at least one side wall has a top edge and a bottom edge, and wherein the end walls extend outwardly between the top edge and the bottom edge.

15. The blade holder according to claim 13, wherein the proximal ends of the elongated members are inserted through the opening in the restrictor.

16. The blade holder according to claim 13, wherein the restrictor has a first side wall and a second side wall, and wherein the interior surfaces of the first and second end walls each has at least one ridge extending between and perpendicular to the first and second side walls.

17. The blade holder according to claim 16, wherein the exterior surface of each of the elongated members has a plurality of transverse ridges, and wherein the ridges are engaged by the at least one ridge on each interior surface of the end walls to secure the restrictor to the elongated members and prevent the elongated members from moving apart.

18. The blade holder according to claim 12, wherein a blade is removably attached to the first and second attachment structures.

19. The blade holder according to claim 12, wherein a flexible blade is removably attached to the first and second attachment structures.

20. The blade holder according to claim 12, wherein a blade is fixedly attached to the first and second attachment structures.

21. The blade holder according to claim 12, wherein a flexible blade is fixedly attached to the first and second attachment structures.

22. The blade holder according to claim 12, wherein the distal ends extend outwardly from the elongated members and form first and second heels on the interior surfaces of the elongated members opposite the distal ends, and wherein the attachment structures are formed in the first and second heels and are opposite to each other.

23. The blade holder according to claim 12, wherein the attachment structures are formed by first and second slots that are adapted to receive the opposing ends of the blade.

24. A blade holder comprising:
first and second elongated members having first and second proximal ends, first and second exterior surfaces and first and second opposing interior surfaces, wherein the proximal ends are connected and the elongated members divergently extend from the proximal ends to first and second distal ends, wherein the first and second elongated members are separated by a distance and the distance increases as the members extend from the proximal ends to the distal ends;
first and second attachment structures located near the first and second distal ends, respectively, wherein the attachment structures are adapted to secure a flexible blade; and
a restrictor comprising first and second end walls having first and second interior surfaces and at least one side wall connecting the first and second end walls and defining an opening, wherein the first and second interior surfaces of the end walls contact the first and second exterior surfaces of the elongated members, respectively, wherein the restrictor is removably mounted on the exterior surfaces of the elongated members, and wherein the restrictor adjusts and affixes the distance between the first and second elongated members.

25. The blade holder according to claim 24, wherein the at least one side wall has a top edge and a bottom edge, and wherein the end walls extend outwardly between the top edge and the bottom edge.

26. The blade holder according to claim 24, wherein the restrictor has a first side wall and a second side wall, and wherein the first and second end walls each has at least one ridge extending between and perpendicular to the first and second side walls.

27. The blade holder according to claim 26, wherein the exterior surface of each of the elongated members has a plurality of transverse ridges, and wherein the ridges are engaged by the at least one ridge on each interior surface of the end walls to secure the restrictor to the elongated members and prevent the elongated members from moving apart.

28. The blade holder according to claim 24, wherein the flexible blade is removably attached to the first and second attachment structures.

29. The blade holder according to claim 24, wherein the flexible blade is fixedly attached to the first and second attachment structures.
